(19) United States
(12) Patent Application Publication
(10) Pub. No.: US 2005/0234332 A1
(43) Pub. Date: Oct. 20, 2005

(54) Method of computer-assisted ligament balancing and component placement in total knee arthroplasty

(76) Inventor: Stephen B. Murphy, Winchester, MA (US)

Correspondence Address: CHIEF PATENT COUNSEL;SMITH & NEPHEW, INC., 1450 BROOKS ROAD, MEMPHIS, TN, 38116, US

(21) Appl. No.: 11/037,898

(22) Filed: Jan. 18, 2005

Related U.S. Application Data
(60) Provisional application No. 60/536,901, filed on Jan. 16, 2004.

(51) Int. Cl. A61F 2/38; A61B 5/05

(52) U.S. Cl. 600/426; 600/427

(57) ABSTRACT

Systems, methods and processes for computer-assisted soft tissue balancing, including ligament balancing, determining surgical cuts, and positioning or placement of the components of the prosthetic knee during TKR. The improved methods, systems, and processes consider and correlate anatomical landmarks and dynamic interactions of the knee bones and soft tissues. The improved methods, systems and processes resolve several problems related to the prosthetic knee component positioning and soft-tissue balancing during computer-assisted TKR. The improved methods, systems and processes are flexible and versatile, provide reliable recommendations to the surgeon, and improve restoration of the knee function and patient recovery.

### CROSS-REFERENCE TO RELATED APPLICATION

This application claims priority to U.S. Provisional Patent Application Ser. No. 60/536,901 entitled “A New Method of Computer-Assisted Ligament Balancing and Component Placement in Total Knee Arthroplasty” filed on Jan. 16, 2004, the entire content of which is incorporated herein by this reference.

### FIELD OF INVENTION

The invention relates generally to computer-assisted surgical (CAS) systems and methods of their use. More specifically, the invention relates to instrumentation, systems, and processes for proper positioning, and alignment of the prosthetic knee components and proper balancing of soft tissues, including any necessary surgical release or contraction, of the knee ligaments, during computer-assisted total knee replacement (TKR) surgery.

### BACKGROUND

Computer-assisted surgical systems use various imaging and tracking devices and combine the image information with computer algorithms to track the position of the patient's anatomy, surgical instruments, prosthetic components, virtual surgical constructs such as body and limb axes, and other surgical structures and components. The computer-assisted surgical systems use this data to make highly individualized recommendations on a number of parameters, including, but not limited to, patient's positioning, the most optimal surgical cuts, and prosthetic component selection and positioning. Orthopedic surgery, including, but not limited to, joint replacement surgery, is one of the areas where computer-assisted surgery is becoming increasingly popular.

During joint replacement surgery, diseased or damaged joints within the musculoskeletal system of a human or an animal, such as, but not limited to, a knee, a hip, a shoulder, an ankle, or an elbow joint, are partially or totally replaced with long-term surgically implantable devices, also referred to as joint implants, joint prostheses, joint prosthetic implants, joint replacements, or prosthetic joints.

Knee arthroplasty is a procedure for replacing components of a knee joint damaged by trauma or disease. During this procedure, a surgeon removes a portion of one or more knee bones forming the knee joint and installs prosthetic components to form the new joint surfaces. In the United States alone, surgeons perform approximately 250,000 total knee arthroplasties (TKAs), or total replacements of a knee joint, annually. Thus, it is highly desirable to improve this popular technique to ensure better restoration of knee joint function and shortening the patient's recovery time.

The structure of the human knee joint is detailed, for example, in “Questions and Answers About Knee Problems” (National Institute of Arthritis and Musculoskeletal and Skin Diseases (NIAMS) Information Clearinghouse National Institutes of Health (NIH), Bethesda, Md., 2001). The human knee joint includes essentially four bones. The lower extremity of the femur, or distal femur, attaches by ligaments and a capsule to the proximal tibia. The distal femur contains two rounded oblong eminences, the condyles, separated by an intercondylar notch. The tibia and the femur do not interlock but meet at their ends. The femoral condyles rest on the condyles of the proximal tibia. The fibula, the smaller shin bone, attaches just below the tibia and is parallel to it. The patella, or knee cap, is at the front of the knee, protecting the joint and providing extra leverage. A patellar surface is a smooth shallow articular depression between the femoral condyles at the front. Cartilage lines the surfaces of the knee bones, cushions them, and minimizes friction. Two C-shaped menisci, or meniscal cartilage, lie between the femur and the tibia, serve as pockets for the condyles, and stabilize the knee. Knee ligaments connect the knee bones and cover and stabilize the joint. The knee ligaments include the patellar ligament, the medial and lateral collateral ligaments, and the anterior (ACL) and posterior (PCL) cruciate ligaments. The medial collateral ligament (MCL) provides stability to the inner (medial) part of the knee. The lateral collateral ligament (LCL) provides stability to the outer (lateral) part of the knee. The anterior cruciate ligament (ACL), in the center of the knee, limits rotation and the forward movement of the tibia. The posterior cruciate ligament (PCL), also in the center of the knee, limits backward movement of the tibia. Ligaments and cartilage provide the strength needed to support the weight of the upper body and to absorb the impact of exercise and activity. Tendons, such as muscle, and cartilage are also instrumental to joint stabilization and functioning. Some examples of the tendons are popliteus tendon, which attaches popliteus muscle to the bone. Pes anserinus is the insertion of the conjoined tendons into the proximal tibia, and comprises the tendons of the sartorius, gracilis, and semitendinosus muscles. The conjoined tendon lies superficial to the tibial insertion of the MCL. The iliotibial band extends from the thigh down over the knee and attaches to the tibia. In knee flexion and extension, the iliotibial band slides over the lateral femoral epicondyle. The knee capsule surrounds the knee joint and contains lubricating fluid synovium.

A healthy knee allows the leg to move freely within its range of motion while supporting the upper body and absorbing the impact of its weight during motion. The knee has generally six degrees of motion during dynamic activities: three rotations (flexion/extension angulations, axial rotation along the long axis of a large tubular bone, also referred to as interior/exterior rotation, and varus/valgus angulations); and three translations (anterior/posterior, medial/lateral, and superior/inferior).

A total knee arthroplasty, or TKA, replaces both the distal femur and the proximal tibia of the damaged or diseased knee with artificial components made of various materials, including, but not limited to, metals, ceramics, plastics, or their combinations. These prosthetic knee components are attached to the bones, and the existing soft tissues are used to stabilize the artificial knee. During TKA, after preparing and anesthetizing the patient, the surgeon makes a long incision along the front of the knee and positions the patella to expose the joint. After exposing the ends of the bones, the surgeon removes the damaged tissue and cuts, or resects, the portions of the tibial and femoral bones to prepare the surfaces for installation of the prosthetic components.

To properly prepare femoral surfaces to accept the femoral and tibial components of the prosthetic knee, the surgeon needs to accurately determine the position of and perform multiple cuts. The surgeon may use various measuring and indexing devices to determine the location of the cut, and various guiding devices, such as, but not limited to, guides, jigs, blocks and templates, to guide the saw blades to accurately resect the bones. After determining the desired position of the cut, the surgeon usually attaches the guiding device to the bone using appropriate fastening mechanisms, including, but not limited to, pins and screws. Attachment to structures already stabilized relative to the bone, such as intramedullary rods, can also be employed. After stabilizing the guiding device at the bone, the surgeon uses the guiding component of the device to direct the saw blade in the plane of the cut.

After preparation of the bones, the knee is tested with the trial components. Soft-tissue balancing, including any necessary surgical release or contraction of the knee ligaments and other soft tissues, is performed to ensure proper post-operative functioning of the knee. Both anatomic (bone-derived landmarks) and dynamic or kinematic (ligament and bone interactions during the knee movement) data may be considered when determining surgical cuts and positioning of the prosthetic components. After ligament balancing and proper selection of the components, the surgeon installs and secures the tibial and femoral components. The patella is typically resurfaced after installation of the tibial and femoral component, and a small plastic piece is often placed on the rear side, where it will cover the new joint. After installation of the knee prosthesis, the knee is closed according to conventional surgical procedures. Post-operative rehabilitation starts shortly after the surgery to restore the knee's function.

In order to ensure proper post-operative functioning of the prosthetic knee, proper positioning, and alignment of the prosthetic knee components and proper balancing, including any necessary surgical release or contraction, of the knee ligaments, during total knee replacement (TKR) surgery is necessary. Improper positioning and misalignment of the prosthetic knee components, and improper ligament balancing commonly cause prosthetic knees to fail, leading to revision surgeries. This failure increases the risks associated with knee replacement, especially because many patients requiring prosthetic knee components are elderly and highly prone to the medical complications resulting from multiple surgeries. Also, having to perform revision surgeries greatly increases the medical costs associated with the restoration of the knee function. In order to prevent premature, excessive, or uneven wear of the artificial knee, the surgeon must implant the prosthetic device so that its multiple components articulate at exact angles, and are properly supported and stabilized by accurately balanced knee ligaments. Thus, correctly preparing the bone for installation of the prosthetic components by precisely determining and accurately performing all the required bone cuts, and correct ligament balancing are vital to the success of TKR.

Traditionally, the surgeons rely heavily on their experience to determine where the bone should be cut, to select, align and place the knee prosthetic components, and to judge how the knee ligaments should be contracted or released to ensure proper ligament balancing. With the advent of computer-assisted surgery, surgeons started using computer predictions in determining surgical cutting planes, ligament balancing, and selection, alignment and positioning of the prosthetic components. In the conventional TKR methods, anatomical (bone-derived landmarks) and dynamic or kinematic (ligament and bone interactions during the knee movement) data are usually considered separately when determining surgical cuts and positioning of the components of the prosthetic knee. Generally, conventional methods are either excessively weighted toward anatomical landmarks on the leg bones or soft tissue balancing (such as adjustment of lengths and tensions of the knee ligaments). Often, only femoral landmarks are considered when determining femoral component positioning, and only tibial landmarks are considered when determining tibial component positioning. In the conventional techniques, irreversible bone cuts in the knee are usually made prior to considering the dynamic balance of the surrounding soft tissue envelope.

One conventional method of determining the femoral resection depth is anterior referencing, which is primarily focused on placing the femoral component in a position that does not notch or stuff anteriorly. The method largely ignores the kinematics of the tibio-femoral joint. Another conventional method, posterior referencing of the femoral resection depth uses the posterior femoral condyles as a reference for resection, but also ignores the dynamic tissue envelope. As an additional drawback, varus and valgus knee deformities affect the resection depth determination by anterior and posterior referencing.

Determining the resection depth based on the surrounding soft tissue envelope is also problematic. If the resection determination is made before the envelope is adequately released, the resection may be inappropriately placed and, likely, excessive. Generally, ignoring the important anatomical landmarks can result in significant malrotation of the femoral component with respect to the bony anatomy.

Conventional anatomical methods of determining femoral component positioning employ the anatomical landmarks such as epicondylar axes, Whiteside's line, and the posterior condyles. By using these anatomical landmarks and ignoring the state of the soft tissue envelope around the knee, the methods encounter certain limitations. For example, the epicondylar axes rely on amorphous knee structures and, thus, are not precisely reproducible. Typically, several sequential determinations of the epicondylar axis produce differing results. Exposing the condyles to determine the epicondylar axis requires significant tissue resection and increases risks to the patient and healing time. Whiteside's line is based on the orientation of the trochlea and is also not precisely reproducible. Furthermore, the line is not correlated with the bony anatomy and ligaments of the tibio-femoral joint in either flexion or extension.

While easily reproduced, resection of the femur parallel to the posterior femoral condyles is potentially inaccurate because it ignores the dynamic status of the surrounding soft tissue envelope. Further, the deformity and wear pattern of the arthritic knee is incorporated into the decision. For example, varus knees typically have significant cartilage wear in the posterior portion of the medial femoral condyle, while the lateral femoral condyle often has a normal cartilage thickness posteriorly. This results in excessive rotation of the femoral component upon placement. Knees with valgus malalignment and lateral compartment arthrosis typically have full-thickness cartilage loss in the lateral femoral condyle, and under-development, or hypoplasia of the condyle. The use of posterior referencing to determine femoral component rotation typically results in excessive internal rotation of the femoral component.

Determining femoral component rotation based on the surrounding soft tissue envelope is attractive because resection of the femur perpendicular to the tibia at 90° of flexion with the ligaments under distraction assures a rectangular flexion gap. However, this method ignores the anatomy of the femur and the extent of the ligament release. For example, if the knee is severely varus and is inadequately released, then the medial side will remain too tight, which results in excessive external rotation of the femoral component. The opposite problem arises due to inadequate released knees with valgus-flexion contractures.

Several systems and methods of computer-assisted ligament balancing are known. One system and method compares the kinematics of the trial prosthetic joint components installed in a knee joint with the kinematics of the normal joint, and provides the surgeon with the information allowing the balancing of the ligaments of the installed prosthetic joint. A video camera registers and a computer determines the position and orientation of the trial components with respect to each other and the kinematics of the trial components relative to one another, identifies anomalies between the observed kinematics of the trial components and the known kinematics in a normal knee, and then suggests to the surgeon which of the ligaments should be adjusted to achieve a balanced knee. Essentially, the femur and the tibia are cut first, and the knee kinematics are checked after the irreversible bone cuts are made and trial prosthetic components are installed. The method is not suitable for prediction of the optimal bone cuts based on the combination of the anatomic and the kinematic data, and does not employ the combination of such data in prosthetic component positioning and ligament balancing. Furthermore, the method requires the use of the video camera to acquire the images of the installed trial components and employs complex “machine vision” algorithm to deduce the position of the prosthetic components and other landmarks from the images.

Another known method of computer assisted ligament balancing provides for ligament balancing prior to femoral resection and prosthetic component positioning, but relies on using a tensor that is inserted between the femur and the tibia and separates the ends of the tibia and the femur during kinematic testing. The method relies extensively on visual images and surgeon judgment in ligament alignment, selection of the implant geometry and size, and determination of the femoral resection plane, and prosthetic component positioning.

There is an unrealized need for improved systems and methods for computer-assisted soft-tissue balancing, component placement, and surgical resection planning during TKA. Particularly, the field of computer assisted TKA needs improved methods and systems that consider and correlate both anatomical landmarks and dynamic interactions of the knee bones and soft tissues. Systems and methods are also desired that incorporate soft tissue balancing and component placement algorithms for quantitative assessment of the anatomical and dynamic aspects of the human knee and provide recommendations on soft tissue balancing, component selection and/or placement, and propose bone resection planes based on iterative convergence of the anatomical and the dynamical factors. Preferably, the desired systems and methods comprise a logic matrix for quantitative assessment of the state of the knee's soft tissues. Systems and methods are also needed that allow for prosthetic component selection and/or placement, soft tissue balancing, and resection planning in a variety of combinations and sequences, based on the patient's need and the surgeon's preference. There is also a need in the systems and methods that allow for component selection and/or placement, soft tissue balancing, and resection planning prior to making any surgical cuts.

In general, there is a need for systems and methods that are flexible and allow the surgeon to operate in accordance with the patient's need and the surgeon's own preferences and experience, that do not limit the surgeon to a particular surgical technique or method, and that allow for easy adaptation of the existing surgical techniques and method to computer-assisted surgery, as well as for the improvement of and development of new surgical techniques and methods. The field of computer-assisted surgery is in need of the improved systems and methods for computer-assisted soft-tissue balancing, component placement, and surgical resection planning during TKA that are versatile, provide reliable recommendations to the surgeon, and result in improved restoration of the knee function and patient's recovery as compared to the conventional methods. Some or all, or combinations of some, of these needs are met in various systems and processes according to various embodiments of the invention.

### SUMMARY

The aspects and embodiments of the present invention provide improved systems, methods and processes for computer-assisted soft tissue balancing, including ligament balancing, such as release or contraction of knee ligaments, determining surgical cuts, and selection and/or positioning or placement of the components of the prosthetic knee during TKR. The improved methods, systems, and processes consider and correlate anatomical landmarks and dynamic interactions of the knee bones and soft tissues. The improved methods, systems and processes resolve several problems related to the prosthetic knee component positioning and soft-tissue balancing during computer-assisted TKR. The improved methods, systems and processes are flexible and versatile, provide reliable recommendations to the surgeon, and improve restoration of the knee function and patient recovery.

In one aspect, certain embodiments of the invention provide a system for use by a surgeon in the course of computer-assisted total arthroplasty on a patient's knee. The system comprises:
at least one first fiducial associated with a femur or a femoral prosthetic component;
at least one second fiducial associated with a tibia or the tibial prosthetic component;
a tracking functionality capable of tracking position and orientation of the at least one first fiducial and the at least one second fiducial;
a computer, wherein the computer is
   adapted to receive and store information from the tracking functionality on the position and orientation of the at least one first fiducial and thus at least one the femur or the femoral prosthetic component, and the at least one second fiducial and thus at least one of the tibia or the tibial prosthetic component,
   adapted to receive and store information acquired during kinematic testing of the knee on the position and orientation of the at least one first fiducial and thus the at least one of the femur or the femoral prosthetic component; and the at least one second fiducial and thus the at least one of the tibia or the tibial prosthetic component;
   adapted to store in memory a logic matrix for assessing kinematics of the knee by comparing to the logic matrix the information acquired during the kinematic testing of the knee, and
   adapted to provide recommendations on soft tissue balancing based on comparison to the logic matrix of the information obtained during the kinematic testing.

The system may further comprise:
an imager for obtaining at least one image of the tibia or the femur, wherein the computer is adapted to receive from the imager and store at least one image of the tibia, the femur, the tibial prosthetic component, or the femoral prosthetic component; and
a monitor adapted to receive information from the computer in order to display the at least one image of the tibia, the femur, the tibial prosthetic component, or the femoral prosthetic component.

The system may further comprise surgical instruments associated with one or more fiducials and adapted for navigation and positioning at the knee. The fiducials associated with the instruments are tracked by the tracking functionality. Real or schematic images of the instruments may be displayed on the monitor.

The systems, methods, and processes provided herein may be adapted to beneficially use the images of the body parts, surgical instrumentations and items, and prosthetic components. Nevertheless, unlike in the existing methods, continuous image acquisition and “machine vision” algorithms are not required for operation of the systems, methods and processes according to certain aspects and embodiments of the present invention. The methods, systems, and processes provided herein are generally adapted to derive the position and orientation of the relevant landmarks and structures by establishing appropriate coordinate systems and tracking the fiducials in relation to the coordinate systems. This advantageously simplifies the operation of the systems, methods and processes of the present invention and releases processing capacity for other operation.

The system may further comprise prosthetic components associated with one or more fiducials and adapted for navigation and positioning at the knee. The fiducials associated with the prosthetic components are tracked by the tracking functionality. Real or schematic images of the prosthetic components may be displayed on the monitor. The computer may be further adapted to store in memory information on various types of prosthetic components, such as their size and mode of positioning, and to provide recommendations to the surgeons on component selection and positioning based on the patient data.

The system may further comprise at least one cutting jig or cutting guide for positioning at the femur or the tibia, wherein the cutting jig is associated with one or more fiducials and the position and orientation of the fiducial associated With the cutting jig is trackable by the computer for navigation and positioning of the cutting jig at the femur. The position of the cutting jig or cutting guide may be adjustable in at least one degree of rotational or at least one degree of translational freedom. The cutting jig or cutting guide may be adapted for performing several surgical cuts.

In another aspect, certain embodiments of the invention provide a method of computer-assisted total arthroplasty on a patient's knee. The method comprises:
registering with a computer at least one first fiducial associated with the femur or the femoral prosthetic component; and at least one second fiducial associated with the tibia or the tibial prosthetic component;
tracking position and orientation of the at least one first fiducial and the at least one second fiducial with a tracking functionality;
using the computer adapted to receive signals and store information from the tracking functionality on the position and orientation of the at least one first fiducial and thus at least one of the femur or the femoral prosthetic component; and the at least one second fiducial and thus at least one of the tibia or the tibial prosthetic component;
assessing performance of the knee using kinematic testing of the knee in six degrees of spatial freedom;
using the computer to compare information from the tracking functionality obtained during the kinematic testing, and
using the computer to provide recommendations on soft tissue balancing of the knee based on the comparison with the logic matrix.

The method may further comprise:
using an imager for obtaining at least one image of a tibia or a femur, wherein the computer is adapted to receive from the imager and store the at least one image of the tibia, the femur, the femoral prosthetic component, or the tibial prosthetic component; and
using a monitor adapted to receive information from the computer to display the at least one image of the tibia, the femur, the tibial prosthetic component, or the tibial prosthetic component.

The method may further comprise registering with the computer and navigating and positioning at the knee of the surgical instruments associated with one or more fiducials. The method may further comprise registering with the computer and navigating and positioning at the knee of prosthetic components associated with one or more fiducials. The method may further comprise registering with the computer and navigating and positioning at the femur, using the images displayed on the monitor, of a cutting jig or a cutting guide associated with one or more fiducials.

Other aspects and embodiments of the present invention extend to an improved versatile and flexible computer algorithm for controlling a computer used during computer-assisted surgery on a patent's knee. When controlling the computer, the algorithm assesses the state of the knee based on the kinematic testing and provides recommendations on soft tissue balancing. The algorithm also allows selection or prosthetic component size, prosthetic component positioning, or planning of surgical cuts, or any combination thereof. The algorithm is adaptable to the patient's needs and the surgeon's preferences and does not limit the surgeon to a particular surgical technique or sequence of steps. The algorithm is easily adaptable to the existing surgical techniques and methods.

Flexibility and versatility are important advantages of certain methods, systems and processes provided by the embodiments of the present invention, unlike existing methods that require the surgeons to perform according to strictly pre-determined procedures and are often limited to a subset of situations that arise in the process of TKA. In contrast, the embodiments of the present invention allow the surgeon to pivot more easily than the conventional methods, taking into account personal preferences, patient's need, and computer generated recommendations.

One embodiment of the invention provided herein is an improved system and method of computer-assisted soft tissue balancing in a knee during total knee arthroplasty, wherein the method considers and correlates both the anatomical landmarks and the dynamic interaction of the knee bones and ligaments. The method advantageously considers both femoral and tibial landmarks. According to some embodiments of the provided method, prosthetic component size, positioning, and surgical cuts can be planned before any irreversible bone cuts are made, although the system and method are adaptable for soft tissue balancing in patients after the surgical cuts are performed, or after the prosthetic components are installed. The method facilitates minimally invasive, small-incision TKR by providing recommendation on optimal surgical cuts and component positioning and reducing the need in revision surgeries.

The system and method register and consider the anatomical landmarks and the dynamic data from the knee in flexion and extension under one or more kinematic tests, such as varus/valgus, AP drawer, and rotation tests. A knee is considered properly balanced when cutting planes advised by the anatomical methods and cutting planes advised by dynamic methods converge. When the anatomic and the dynamic recommendations differ, more soft tissue balancing may be provided, after which the anatomic and the dynamic recommendations may change. This is an iterative process.

An embodiment of a method of computer-assisted soft tissue balancing in a knee during total knee arthroplasty is provided. Essentially, the method establishes a rectangular gap between tibia and femur in both flexion and extension without distorting the anatomy of the knee. It is perfectly conducted after the surgeon exposes the bones, and performs any preliminary osteophyte (bony excrescence at the joint margin, such as those seen in osteoarthritis) resections and ligament release. The method employs the following steps performed with computer assistance:
1. Establishing femoral and tibial coordinate systems by tracking at least one fiducial associated with a femur and at least one fiducial associated with a tibia;
2. Establishing in a computer memory a femoral resection plane perpendicular to a mechanical axis of the femur (an anatomical femoral resection plane), and a proposed tibial resection plane perpendicular to a mechanical axis of the tibia.
3. Placing the knee under distraction in flexion and extension in at least one of varus/valgus, AP drawer, or rotation tests, and establishing, in flexion and extension, in a computer memory femoral resection planes perpendicular to the long axis of the tibia.
4. Comparing the femoral resection planes perpendicular to the long axis of the tibia (dynamic resection planes) to the femoral resection planes perpendicular to the mechanical axis of the femur (anatomical resection planes), whereby the state of the ligament balance of the knee is represented in flexion and extension by an angle formed between the femoral anatomical resection plane and the femoral dynamic resection planes in flexion and extension.
5. Using the computer to provide recommendations to the surgeon on adjustment of soft tissue leading to the decrease of the angle formed between the femoral anatomical resection plane and the femoral dynamic resection planes in flexion and extension.
6. Adjusting the soft tissues; and
7. Repeating the steps 4-6 until the anatomical and the dynamic planes converge.

The method may further comprise the steps of placing a distal femoral cutting jig at the femur and resecting the femur based at the recommended converged planes.

Various embodiments of the present invention are better understood in reference to the following schematic drawings that are provided herein for illustrative purposes and are in no way limiting. The embodiments of the present invention may differ from the provided schematic illustrations.

### BRIEF DESCRIPTION OF THE DRAWINGS

FIG. 1 is a schematic representation of an operation of a data input devices during computer assisted surgery.

FIG. 2 shows a knee during computer assisted TKA after preliminary osteophyte resection and ligament release.

FIG. 3 is a schematic representation of improved soft tissue balancing algorithm according to a preferred embodiment of the invention.

FIG. 4 is a schematic representation of anatomical landmarks used in kinematic assessment of the knee, wherein the extended knee is shown in the anterior/posterior direction.

FIG. 5 is a schematic representation of anatomical landmarks used in kinematic assessment of the knee, wherein the extended knee is shown in the medial/lateral direction. Comment above

FIG. 6 is a schematic representation of anatomical landmarks used in kinematic assessment of the knee, wherein the flexed knee is shown in the anterior/posterior direction. Comment above

FIG. 7 is a schematic representation of anatomical landmarks used in kinematic assessment of the knee, wherein the flexed knee is shown in the medial/lateral direction. Comment above

FIG. 8 is a schematic representation of anatomical and dynamic resection planes in a knee at full extension.

FIG. 9 is a schematic representation of anatomical and dynamic resection planes in a flexed knee.

### DETAILED DESCRIPTION

Various aspects and embodiments of the present invention provide improved systems, methods and processes of soft tissue balancing, determining surgical cuts, and positioning of the components of the prosthetic knee during computer-assisted TKA. During installation of a prosthetic knee, systems according to certain embodiments of the present invention advantageously assess and provide feedback on the state of the soft tissues in a rage of motion, such as under varus/valgus, anterior/posterior and rotary stresses, and can suggest or at least provide more accurate information than that obtainable by the conventional methods about soft tissue adjustments, including, but not limited to the recommendations on which ligaments the surgeon should release or contract in order to obtain correct balancing, alignment and stability of the knee joint.

Systems, methods and processes according to various aspects and embodiments of the present invention can also provide recommendations on implant size, positioning, and other parameters relevant to achieving optimal kinematics of the knee joint. As used herein, the term “kinematics” means the pattern of motion having six degrees of freedom. More particularly, the term “kinematics” in reference to a knee joint is used to denote the motion, or articulation, of the knee joint in six degrees of freedom. Systems and processes according to various embodiments of the present invention can also include databases of information or logic matrixes regarding tasks such as soft tissue balancing, in order to provide suggestions to the surgeon based on performance the knee in kinematic tests.

The tests, such as varus/valgus knee distraction, AP drawer test, or axial rotation are known. Tests which are presently unknown can be included in systems and processes according to the invention in the future. When the knee is distracted in the course of kinematic testing, a physical spacer or tensor, such as an inflatable balloon, a hydraulic bag, a mechanical device, or any other physical tensor or spacer, may be applied to the to the knee to achieve the degree of tension that is the closest to the normal knee tested this way. For example, for AP drawer test, the spacer is applied to the medial side to achieve a desired degree of tension. The physical spacer is typically adapted to be locked or stabilized in any desired position. The spacer may comprise a measurement scale to allow a reading of the gap obtained, and may be adapted to feed the information to the computer functionality for display and/or use as desired. Nevertheless, it is one advantage of the present invention over the existing methods that the use of the spacers and tensors is optional and is based on the surgeon's consideration and patient's need.

### Computer-Assisted Surgical Systems

In one aspect, certain embodiments of the present invention provide a computer-assisted surgical system for use by a surgeon during TKA. Generally, computer-assisted surgical systems use various imaging and tracking devices and combine the image information with computer algorithms to track the position of the patient's anatomy, surgical instruments, prosthetic components, virtual surgical constructs such as body and limb axes, and other surgical structures and components. Some of the computer-assisted surgery systems use imaging systems based on CT scans and/or MRI data or on digitized points on the anatomy. Other systems align preoperative CT scans, MRIs, or other images with intraoperative patient positions. A preoperative planning system allows the surgeon to select reference points and to determine the final implant position. Intraoperatively, the computer-assisted surgery system calibrates the patient position to that preoperative plan, such as by using a “point cloud” technique, conventional kinematic techniques, and/or a robot to make bone preparations. Other systems use position and/or orientation tracking sensors, such as infrared sensors acting stereoscopically or otherwise, to track positions of body parts, surgery-related items such as implements, instrumentation, trial prosthetics, prosthetic components, and virtual constructs or references such as rotational axes which have been calculated and stored based on designation of bone landmarks.

As used herein, the term “position and orientation” denotes a position of an object in three-dimensional space with respect to all six degrees of freedom relative to a known coordinate system. When the object, such as a body part or a prosthetic component, is a solid member, and because the position and orientation of the fiducial marks associated with the targets are fixed, by knowing the position and orientation of the fiducials in space, the position and orientation of all surfaces on the object is also known. If the position and orientation of both femoral and tibial prosthetic components is known with respect to a single reference system, the position and orientation of the components relative to one another may be determined. Prosthetic components can be navigated relative to each other in an absolute fashion, that is the computer assumes that the trials are positioned perfectly, and the gaps between the components are tracked relative to each other without the need for landmarking and without fiducials applied to the tibia and the femur. Additional landmarking, for example, for validation purposes, can be additionally be performed (for example, relative to the location of head of the femur and center of the ankle) to determine that the components were placed as desired.

Processing functionality, whether standalone, networked, or otherwise, takes into account the position and orientation information as to various items in the position sensing field (which may correspond generally or specifically to all or portions or more than all of the surgical field) based on sensed position and orientation of their associated fiducials or based on stored position and/or orientation information. The processing functionality correlates this position and orientation information for each object with stored information regarding the items, such as a computerized fluoroscopic imaged file of a bone, a wire frame data file for rendering a representation of an instrumentation component, trial joint prosthesis or actual joint prosthesis, or a computer generated file relating to a rotational axis or other virtual construct or reference. The processing functionality then displays position and orientation of these objects on a screen or monitor, or heads-up display or otherwise. The surgeon may navigate tools, instrumentation, prosthetic components, actual prostheses, and other items relative to bones and other body parts to perform a surgery more accurately, efficiently, and with better alignment.

The computer-assisted surgical systems use the position and orientation tracking sensors to track the fiducial or reference devices associated with the body parts, surgery-related items such as implements, instrumentation, trial prosthetics, prosthetic components, and virtual constructs or references, such as limb rotational axes calculated and stored based on designation of bone landmarks. Any or all of these may be physically or virtually associated with any desired form of mark, structure, component, or other fiducial or reference device or technique that allows position or orientation, or both, of the associated item to be sensed and tracked in space, time, or both. Fiducials can be single markers or reference frames or arrays containing one or more reference elements. Reference elements can be active, such as energy emitting, or passive, such as energy reflective or absorbing, or any combination thereof. Reference elements may be optical, employ ultrasound, or employ any suitable form of electromagnetic energy, such as infrared, micro or radio waves. In general, any other suitable form of signaling may also be used, as well as combinations of various signals. To report position and orientation of the item, the active fiducials, such as microchips with appropriate field or a position/orientation sensing functionality, and a communications link, such as a spread-spectrum radio frequency link, may be used. Hybrid active/passive fiducials are also possible. The output of the reference elements may be processed separately or in concert by the processing functionality.

To locate and register an anatomical landmark, a CAS system user may employ a probe operatively associated with one or more fiducials. For example, the probe may be is triangulated in space relative to two sets of fiducials. The one or more fiducials provide information relating the landmark via a tracking/sensing functionality to the processing functionality. To indicate input of a desired point to the processing functionality, one or more devices for data input are commonly incorporated into the computer-assisted surgery systems. The data input devices allow the user to communicate to the processing functionality to register data from the probe-associated fiducials.

A CAS system user may input data to the computer functionality by a variety of means. Some systems employ a conventional computer interface, such as a keyboard or a computer mouse, or a computer screen with a tactile interface. In some systems, the user presses a foot pedal to indicate to the computer to input probe location data. Others use a wired keypad or a wireless handheld remote. The probe may also interact with arrays, sensors, or a patient in such a way as to act like an input device.

During surgery, CAS systems employ a processing functionality, such as a computer, to register data on position and orientation of the probe to acquire information on the position and orientation of the patient's anatomical structures, such as certain anatomical landmarks, for example, a center of a femoral head. The information is used, among other things, to calculate and store reference axes of body components such as in a knee or a hip arthroplasty, for example, the axes of the femur and tibia, based on the data on the position and/or orientation of the improved probe. From these axes such systems track the position of the instrumentation and osteotomy guides so that bone resections position the prosthetic joint components optimally, usually aligned with a mechanical axis. Furthermore, the systems provide feedback on the balancing of the joint ligaments in a range of motion and under a variety of stresses and can suggest or at least provide more accurate information than in the past about the ligaments that the surgeon should release in order to obtain correct balancing, alignment and stability of the joint, improving patient's recovery. CAS systems allow the attachment of a variable adjustor module so that a surgeon can grossly place a cutting block based on visual landmarks or navigation and then finely adjust the cutting block based on navigation and feedback from the system.

CAS systems can also suggest modifications to implant size, positioning, and other techniques to achieve optimal kinematics. Instrumentation, systems, and processes according to the present invention can also include databases of information regarding tasks such as ligament balancing, in order to provide suggestions to the surgeon based on performance of test results as automatically calculated by such instrumentation, systems, and processes.

CAS systems can be used in connection with computing functionality that is networked or otherwise in communication with computing functionality in other locations, whether by PSTN, information exchange infrastructures such as packet switched networks including the Internet, or as otherwise desired. Such remote imaging may occur on computers, wireless devices, videoconferencing devices or in any other mode or on any other platform which is now or may in the future be capable of rending images or parts of them produced in accordance with the present invention. Parallel communication links such as switched or unswitched telephone call connections or Internet communications may also accompany or form part of such telemedical techniques. Distant databases such as online catalogs of implant suppliers or prosthetics buyers or distributors or anatomical archives may form part of or be networked with the computing functionality to give the surgeon in real time access to additional options for implants which could be procured and used during the surgical operation.

In some aspects and embodiments, the present invention relates to a system for use by a surgeon during TKA, comprising: a tracking functionality adapted to track position and orientation of at least one fiducial attached to a knee bone; a computer adapted to receive information from the tracking functionality in order to track position and orientation of the fiducials, and instruments for release and contraction of the knee ligaments. The system may further comprise a tensor for applying tension to the knee ligaments after resection of the patients' femur or tibia. The computer is adapted to store a logic matrix with the various kinematic parameters of the knee. The computer is programmed to compare the patient's knee kinematic data obtained by the surgeon during kinematic testing with the parameter stored in the logic matrix and to issue the recommendations to the surgeon regarding release or contraction of the knee ligaments. The computer may also be adapted to store the data on the anatomical landmarks, the data relating to the three dimensional position and orientation of the knee prosthetic components, and the data on the potential or existing surgical resection planes. The computer may also be adapted to calculate virtual surgical constructs, such as the surgical resection planes or the axes, based on the data stored in the memory.

### Minimally Invasive Surgery

In one more aspect, the embodiments of the present invention provide a computer-assisted surgical system for TKA that is particularly useful, although not limited to, minimally invasive surgical applications. The term “minimally invasive surgery” (MIS) generally refers to the surgical techniques that minimize the size of the surgical incision and trauma to tissues. Minimally invasive surgery is generally less intrusive than conventional surgery, thereby shortening both surgical time and recovery time. Minimally invasive TKA techniques are advantageous over conventional TKA techniques by providing, for example, a smaller incision, less soft-tissue exposure, improved collateral ligament balancing, and minimal trauma to the extensor mechanism (see, for example, Bonutti, P. M., et al., Minimal Incision Total Knee Arthroplasty Using the Suspended Leg Technique, Orthopedics, September 2003). To achieve the above goals of MIS, it is necessary to modify the traditional implants and instruments that require long surgical cuts and extensive exposure of the internal knee structures. Minimally invasive techniques are advantageous over conventional techniques by providing, for example, a smaller incision, less soft-tissue exposure, and minimal trauma to the tissues. To achieve the above goals of MIS, it is necessary to modify the traditional surgical techniques and instruments to minimize the surgical cuts and exposure of the patient's tissues.

### System and Methods for Use by a Surgeon During TKA

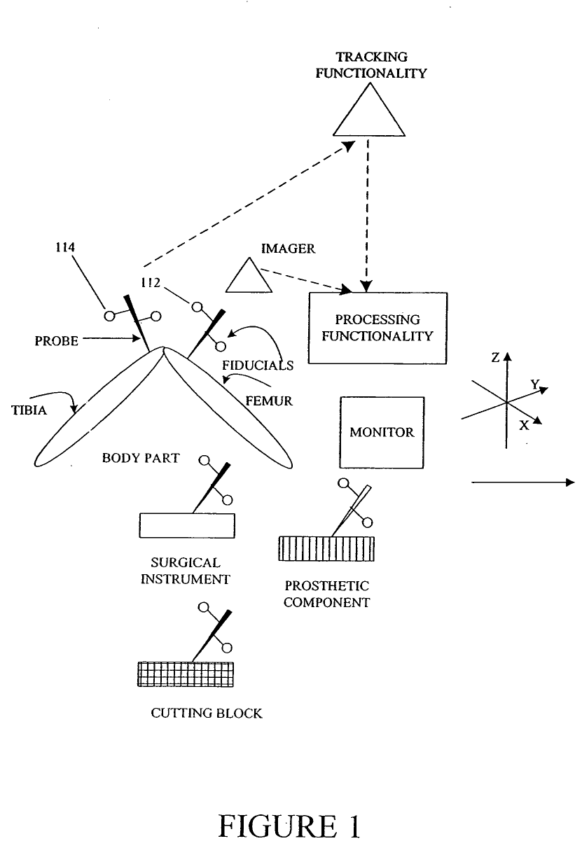

In one aspect, the invention provides a system for use by a surgeon in the course of computer-assisted total arthroplasty on a patient's knee. FIG. 1 is a schematic view showing one embodiment of a system according to the present invention. According to this embodiment, the system is used to perform a knee surgery, particularly total knee arthroplasty. In reference to FIG. 1, the system comprises a fiducial associated with the femur or the femoral prosthetic component; a fiducial associated with the tibia or the tibial prosthetic component; a tracking functionality capable of tracking position and orientation of the femoral and the tibial fiducial. The system can track various body parts, such as tibia and femur, or prosthetic components, to which fiducials are implanted, attached, or otherwise associated with physically, virtually, or otherwise. In the embodiment shown in FIG. 1 fiducials are structural frames, at least some of which comprise reflective elements, LED active elements, or both, for tracking using a tracking functionality, comprising one or more stereoscopic position/orientation sensors, such as infrared sensors. The sensors are adapted for sensing, storing, processing and/or outputting data relating to position and orientation of the fiducials and, thus, components with which they are associated.

The system according to this embodiment of the present invention also comprises a computer comprising a processing functionality generally adapted to receive and store information from the tracking functionality on the position and orientation of the femoral fiducial (112) and the tibial fiducial (114). In the embodiment shown in FIG. 1, the computer may include a processing functionality, a memory functionality, an input/output functionality, on a standalone or distributed basis, via any desired standard, architecture, interface and/or network topology. In this embodiment, computer functionality is connected to a monitor, on which graphics and data may be presented to the surgeon during surgery. The screen may comprise a tactile interface so that the surgeon may point and click on screen. The system may also comprise a keyboard interface, a mouse interface, a voice recognition functionality, a foot pedal, or any other functionality for imputing information, wired or wireless, or any combination or modification of the functionalities. Such functionalities allow the system's user, such as, but not limited to, a nurse or a surgeon, to control or direct the functionality, among other things, to capture position/orientation information.

Items such as body parts, virtual surgical constructs, prosthetic components, including trial components, implements, and/or surgical instrumentation may be tracked in position and orientation relative to body parts using fiducials. Computer functionality can process, store, and output various forms of data relating to position, configuration, size, orientation, and other properties of the items. When they are introduced into the field of tracking functionality, computer functionality can generate and display separately or in combination with the images of the body parts computer-generated images of body parts, virtual surgical constructs, trial components, implements, and/or surgical instrumentation, or other items for navigation, positioning, assessment or other uses.

To perform TKA according to aspects and embodiments of the present invention, surgically related items, as well as body parts, items of the anatomy and virtual surgical construct are registered, which means ensuring that the computer know which body part, item, or constructs corresponds to which fiducial or fiducials, and how the position and orientation of the body part, item, or construct is related to the position and orientation of its corresponding fiducial. Registration of body parts may occur in conjunction with acquisition of images, which can be obtained together with position and/or orientation information received by, noted and stored within the computer functionality. Registration of body parts may also occur independently from acquisition of images. The images may aid the user in designating various anatomical landmarks. For example, the center of the femoral head may be designated with the purpose of establishing the mechanical axis of the leg. The center of rotation can be established by articulating the femur within the acetabulum to capture a number of samples of position and orientation information, from which the computer may calculate the center of rotation. The center of rotation can also be established by using the probe and designating a number of points on the femoral head and thus allowing the computer to calculate the center. Graphical representations and schematics, such as controllably sized circles displayed on the monitor and fitted by the surgeon to the shape of the femoral head can also be used to designate the center of the femoral head. Nevertheless, the systems according to the aspects and embodiments of the present invention do not necessarily rely on images to designate the anatomical landmarks and surgical axes. Other techniques for determining, calculating or establishing points or constructs in space can be used in accordance with the present invention.

Before or after registering the body parts, the surgical items may also be designated by instructing the computer to correlate the data corresponding to a particular fiducial or fiducials with the data need to represent a particular surgical item. The computer then stores identification, position and orientation information relating to the fiducial or fiducials correlated with the data for the registered surgical item. Upon registration, when sensor tracks the item, the monitor can show the item, moving and turning properly positioned and oriented, relative to the body part which is also being tracked. The user may navigate the shown item.

Similarly, various virtual surgical constructs may be registered, such as the mechanical axis of the leg that passes through the rotational center of the hip and the rotational center of the ankle, the mechanical axis of the femur that passes through the rotational center of the hip and the center of the femoral condyles, or the mechanical axis of the tibia, that passes through the rotational center of the ankle and the center of the tibial plateau. Using the images and/or the probe, the surgeon can select and register in the computer the center of the femoral head and ankle in orthogonal views on a touch screen. The surgeon then uses the probe to select any desired anatomical landmarks or references at the operative site of the knee or on the skin or surgical draping over the skin. These points are registered in three-dimensional space by the system and tracked relative to the fiducials on the patient anatomy, which are preferably placed intraoperatively.

Registering points using actual bone structure is one preferred way to establish the axis, but other methods can be employed, such as a cloud of points approach by which the probe is used to designate multiple points on the surface of the bone structure, as can moving the body part and tracking movement to establish a center of rotation as discussed above. Once the center of rotation for the hip, the center of rotation of the ankle, the condylar components or the tibial plateau are registered, the computer is able to calculate, store, and render, or otherwise use the data related to these anatomical landmarks.

One aspect of the present invention ensures that the prosthetic components are positioned for the best possible balance of soft tissues in the knee. Another aspect of the present invention ensures that the prosthetic components of the correct size and type are chosen to achieve the best possible balance of soft tissues in the knee. Thus, the methods, systems and processes of the present invention may be adapted to provide recommendations on the prosthetic component type and size, as well as on its positioning. If needed, additional components or parts may be installed to improve the position of the implant. Such need may particularly arise during revision surgeries, when significant portions of the bony anatomy have been removed. Pre-calibrated trial prosthetic components, such as trial prosthetic components adapted for calibration can be utilized in the systems and processes according to the embodiments of the present invention. Calibration ensures that accuracy of the stored in the computer memory data on the geometry of the component, and its position and/or orientation relative to the associated one or more fiducials.

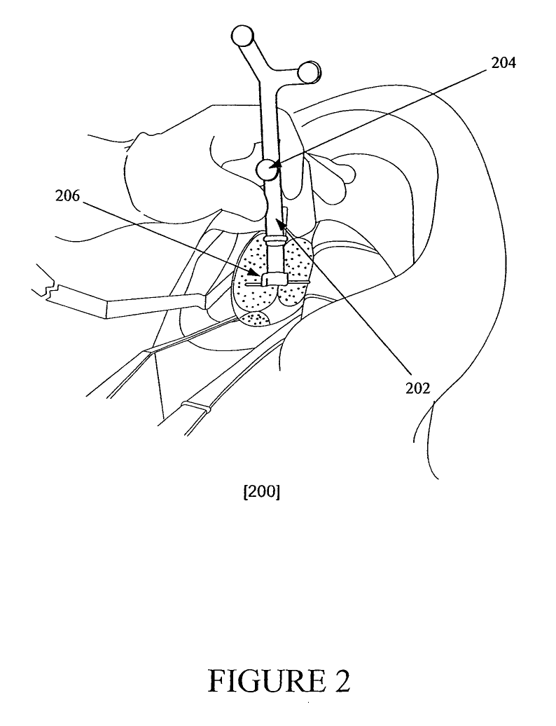

FIG. 2 shows an exposed human knee (200) in a surgical field after the osteophyte resection and the preliminary ligament release. The user registers the anatomical landmarks by using a probe (202) comprising fiducials (204) and associated with the distal femur (206).

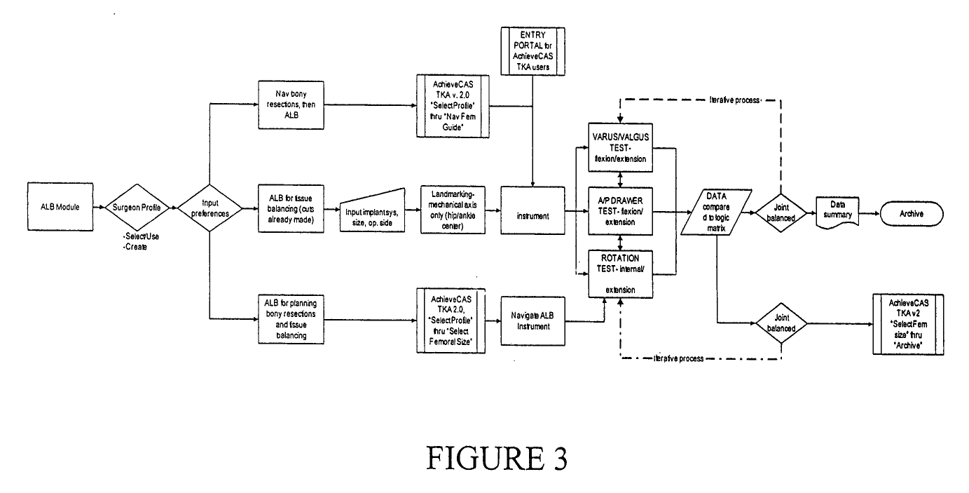

FIG. 3 schematically represents the improved soft-tissue balancing algorithm according to certain embodiments of the preset invention. During operation of these improved systems, methods and processes according to these aspects and embodiments of the present invention, the user, such as a surgeon, commands the computer to retrieve the soft-tissue balancing algorithm, also referred to as advanced ligament balancing algorithm, ligament balancing algorithm, or ALB. It is to be understood that the term “ligament balancing” as used herein may refer to testing and adjustment of the soft tissues of the knee, including, but not limited to, ligaments, tendons, and knee capsule soft tissues. Upon retrieval of the algorithm by the computer, the surgeon enters his or her profile and preferences into the computer memory, or commands the computer to retrieve a profile from its memory. The algorithm takes into account the stored profile and preferences when providing recommendations and feedback on soft tissue balancing.

The surgeon then selects the appropriate option for soft tissue balancing. In a preferred embodiment, the algorithm provides at least the following options: soft tissue balancing and prosthetic component placement in a knee, wherein the tibial or femoral, or both, bone cuts have previously been performed, such as after prosthetic implant installation or during revision surgery; navigation of bony resections in a knee followed by component placement and soft tissue balancing; and soft tissue balancing, component placement, and bony resection planning in a knee.

In one embodiment, described herein in reference to FIG. 3, the user employs the system and method provided herein for soft tissue balancing. For example, the user employs the balancing algorithm in a knee where the surgical cuts have been performed. The trial prosthetic components can also have been selected and installed utilizing conventional surgical methods. When using the balancing algorithm for ligament and soft tissue balancing and prosthetic component placement in a knee where the tibial or femoral, or both, bone cuts have been performed, the surgeon establishes femoral and tibial coordinate systems, inputs or invokes from computer memory the implant and surgical data, such as, but not limited to, implant type, size, the operated on side of the patient. In this embodiment, one or more fiducials can be associated with the prosthetic components, such as a femoral trial prosthetic component, a tibial trial prosthetic component, or both. In this case, the femoral and tibial coordinate systems are defined, at least in part, by the prosthetic component geometry. The surgeon can also establish surgical axes using existing anatomical landmarks. One such axis is the mechanical axis of the leg that passes through rotational centers of the hip and the ankle center. Various procedures are known and may be employed to establish the mechanical axis. Using the existing anatomical landmarks allows the system to determine the position and orientation of the surgical components in relation to the existing landmarks and provides the beneficial information for verification and/or adjustment of the prosthetic component placement. Using the navigated trial components can eliminate the need for fiducial placement on the femur and the tibia, thus eliminating the stress-concentrations caused by fiducial fixation. The navigational algorithm is invoked for computer-assisted navigation of the prosthetic components and surgical instruments at the knee. The system uses the known location, such as, but not limited to, full extension, neutral rotation, and neutral rollback, to acquire knee gap data prior to kinematic testing. The surgeon then performs kinematic testing at the flexed and extended knee. The kinematic tests include but are not limited to, varus/valgus rotation, anterior/posterior drawer, and internal/external rotation. The tests are conventional in the field of orthopedic surgery and are performed according to the accepted in the field guidelines. Other tests can also be used. The computer registers the anatomical reference points at the distal femoral and proximal tibial surfaces, and calculates the kinematic parameters based on the relative positions of the reference points.

In another embodiment, the systems and methods provided herein allow the user, such as a surgeon, to navigate surgical cuts after anatomical landmarking is performed, and balance the soft tissues after the cuts have been made. In further reference to FIG. 3, when using the advanced soft tissue balancing algorithm for navigation of bony resections in a knee, followed by component placement and ligament and tissue balancing, the surgeon establishes femoral and tibial coordinate systems and the surgical references using the existing anatomical landmarks at the distal femur and proximal tibia. For example, tibial and femoral fiducials are applied the tibia and the femur, the head of the femur is identified, the center of the ankle is identified, and other landmarking is performed as desired, such as determination of rotational axes, to establish the anatomical parameters used in determining bony cuts for prosthetic component placement. The navigational algorithm is invoked to navigate the surgical instruments, cutting jigs and guides, and prosthetic components. The surgeon performs the resections, selects and navigates prosthetic components, and places them at the knee. Following component placement, the surgeon performs kinematic testing at the flexed and extended knee. The kinematic tests include but not limited to, varus/valgus rotation, anterior/posterior drawer, and internal/external rotation. The computer registers the anatomical reference points at the distal femoral and proximal tibial surfaces, and calculates the kinematic parameters based on the relative positions of the reference points.

The embodiment of the system and method provided herein can be adapted to employ any number of instruments to navigate the surgical space for ligament and soft tissue balancing. Non-navigated prosthetic components, including trial prosthetic components, also commonly referred to as trials, spacer blocks, and tensioners can also be used, particularly, but not limited to, during testing and logic matrix comparison. Navigated trial components can be used, providing an additional advantage of confirming the location of the trials relative to the cuts. Navigated cutting blocks could remain in place, or a lock feature could be employed so that the system is able to determine where the cuts are relative to the instruments in the space. If non-navigated instruments are used, prior to testing, the system can acquire knee gap data in a known position, for example, but not limited to, full extension, neutral rotation, and neutral rollback.

In further reference to FIG. 3, when using the ligament balancing algorithm for a ligament and soft tissue balancing, component placement, and surgical resection planning in a knee, the surgeon establishes femoral and tibial coordinate systems and the surgical references using existing anatomical landmarks. The navigation algorithm is invoked to navigate the surgical instruments used in soft tissue balancing. The surgeon performs kinematic testing at the flexed and extended knee. The kinematic tests include but not limited to, varus/valgus rotation, anterior/posterior drawer, and internal/external rotation. The computer registers the anatomical reference points at the distal femoral and proximal tibial surfaces, and calculates the kinematic parameters based on the relative positions of the reference points.

The embodiments of the system and method provided herein compare data acquired during the kinematic testing of the patient's knee to baseline kinematic data. This comparison is referred to as a logic matrix or logic chart, schematically illustrated in Table 2. As stated earlier, surgeon traditionally rely on their judgment during soft tissue balancing and often use subjective measures to balance the knee joint. The aspects of the present invention provide an objective assessment of the state of the balance of the knee by determining the gaps between the femur and the tibia at full flexion and full extension and at intervals in between as desired during diagnostic varus/valgus, AP drawer, and rotational tests. The system analyzes the gap data, and compares the data to a logic matrix. For example in the case of varus/valgus testing, if the gap data, or the distances between the medial and the lateral femur and tibia, are below the thresholds stored in the logic matrix, the system reports a normal knee balance and indicates that no soft tissue needs to be balanced. However, if the gap distances on the medial and/or lateral side exceed the threshold values stored in the logic matrix, then system directs the user's attention to the compartment that appears to be imbalanced and suggest that the user evaluates those soft tissue structures. For example, after the user has acquired data from AP drawer, varus/valgus, and rotational testing, the system indicates that the knee appears to be tight medially in flexion only, and that the user should evaluate the anterior medial collateral ligament and perform releases deep or superficially as appropriate.

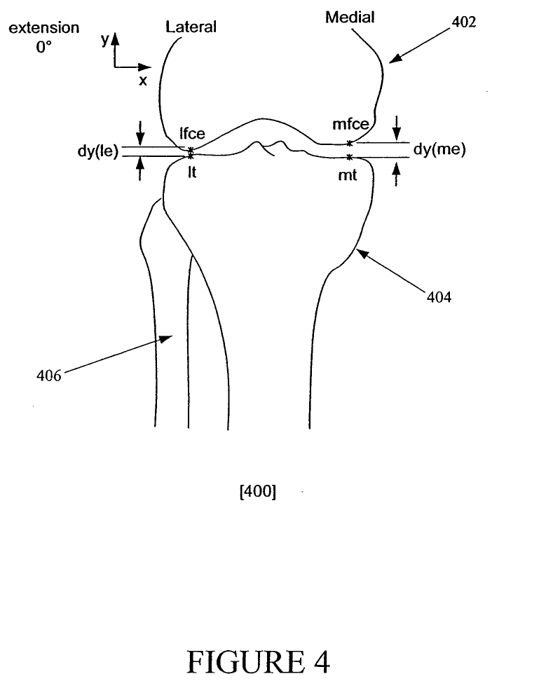

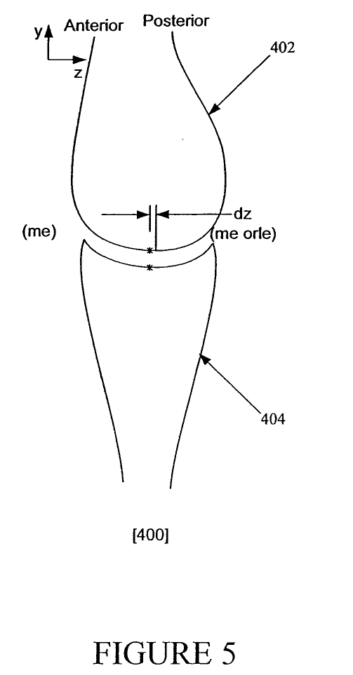

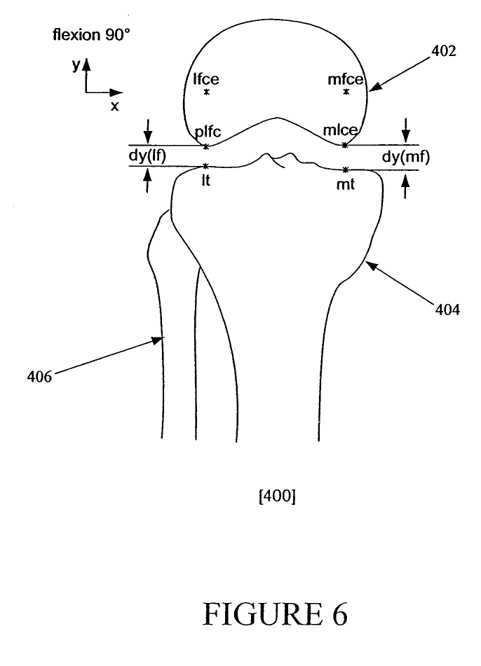

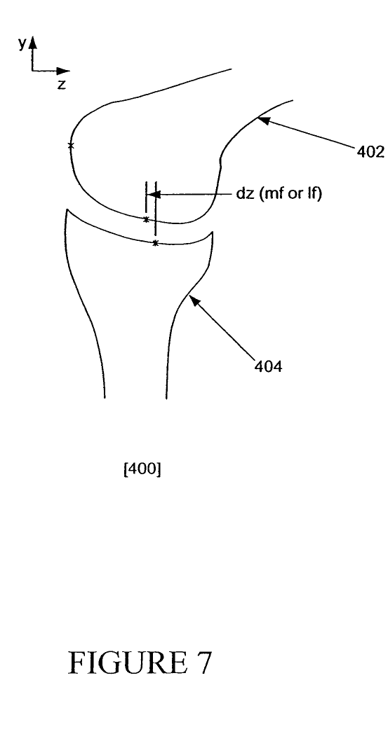

FIGS. 4-7 schematically illustrate a human knee in extension (FIGS. 4 and 6) and flexion (5 and 7) the kinematic parameters and variables registered and/or calculated during kinematic testing and the anatomical reference points used in the calculation of the parameters. For ease of description, the knee (400), comprising femur (402), tibia (404) and fibula (406) is shown with respect to Cartesian coordinates. In FIGS. 4 and 6 (a view in the anterior-posterior direction), the x- and y-axes lie in a horizontal plane, and the z-axis extends vertically. In FIGS. 5 and 7 (a view in the medial-lateral direction), the y- and z-axes lie in a horizontal plane, and the x-axis extends vertically. Thus, dx represents the distance in x direction (medial/lateral); dy represents the distance in y direction (proximal/distal); dz represents the distance in z direction (anterior/posterior). However, it will be appreciated that this method of description is for convenience only and is not intended to limit the invention to any particular orientation. Likewise, unless otherwise stated, terms such as “top,” “bottom ,” “upper,” “lower,” “left,” “right,” “front,” “back,” “proximal,” “distal,” “medial,” “lateral,” “inferior,” “superior” and the like are used only for convenience of description and are not intended to limit the invention to any particular orientation. The anatomic reference points and the kinematic parameters, or variables, used during soft tissue balancing, include, but are not limited to, those listed in Table 1.

TABLE 1
Kinematic variables

| Variable | Description |
|---|---|
| ri | internal rotation |
| re | external rotation |
| fa | flexion angle |
| lfce | lateral femoral condyle tangent point in extension |
| mfce | medial femoral condyle tangent point in extension |
| lt | lateral tibial tangent point in extension and flexion |
| mt | medial tibial tangent point in extension and flexion |
| plfc | posterior lateral femoral condyle tangent point in |
| | flexion |
| pmfc | posterior medial femoral condyle tangent point in |
| | flexion |
| le | distance from lfce to lt (in extension) |
| me | distance from mfce to mt (in extension) |
| lf | distance from plfc to lt (in flexion) |
| mf | distance from pmfc to mt (in flexion) |

It is to be understood that the reference points used in the assessment of the kinematic parameters do not have to be repeatedly registered and/or tracked during the kinematic testing. Once the patient's tibia and femur are registered by or known to the computer-assisted surgical systems, the system tracks the one or more fiducials associated with the tibia and the femur, the femoral or tibial prosthetic components, or any combination thereof, respectively, and deduces the location of the reference points from the information on the position/orientation of the tibia and the femur. The position and orientation of the reference points relative to the corresponding fiducials may be initially saved in the computer memory by inputting their location with an appropriate probe. Alternatively, the position and orientation of the reference point may be deduced from the position of the tracked fiducials based on the tibial and femoral surface data stored in the computer memory.

Table 2 (A and B) schematically shows an embodiment of a logic matrix used for assessment of the state of the knee based on the kinematic testing according to one embodiment of the invention. It is to be understood that Table 2 is divided into parts A and B for ease of representation only. Other information can also be added or deleted to or from the matrix, and the information can be included in the matrix in any desired format, with any desired arrangement of cells, and any desired context and format of information in these. In any event, the logic matrix according to the embodiment generally relates the results of the kinetic testing in a knee (columns D through I), their causes (column C) , and associated conditions (column A). As shown in columns D through I of Table 2 (A and B), the computer assesses and/or compares the kinematic parameters that are registered and calculated during the kinematic tests listed in row 1, columns D through I. Using the criteria shown in columns D though 1, rows 2 through 22, the computer evaluates the results of the kinematic tests against the logic matrix Based on the relationships in the logic matrix, the computer outputs the causes (column A) and the soft tissues needing adjustments (column C). The computer can output specific instructions, if desired, such as to release a certain ligament, or other action. These instructions can also be included in the matrix if desired. The logic matrix may be expanded or otherwise changed as desired and/or as more surgical data are collected, in order to incorporate various parameters and criteria, associated causes and conditions, kinematic tests, and so on. Based on the causes and conditions identified by the computer, the surgeon adjusts the soft tissues, and repeats the testing cycle, followed by the comparison to the logic matrix. The iterative cycle of the kinematic testing, comparison to the logic matrix and ligament balancing by the surgeon continues until reasonable convergence of the results of the kinematic testing with the desirables kinematic properties stored in the computer memory. This process preferably results in the improved balance of the knee joint. It is to be appreciated that the general principles of the iterative convergence methods and their limitations are well known and are employed in certain embodiments of the present invention. For example, the selection of the convergence criteria, assessment of the relative errors, and avoidance of the local optima are routinely addressed in the field of the iterative convergence methods and are attended to as relevant and according to the conventional procedures.

When improved balance of the knee joint is achieved, the surgery may be completed according to the conventional methods and surgical data summary may be stored in the computer memory, for example, for archival purposes. The data may also be used intraoperatively to provide recommendations to the surgeon on the optimal resection planes and the surgeon may perform resections de novo, followed by component selection and placement, or improve on the preliminary resections based on the recommendations provided by the system.

TABLE 2
Logic matrix

| | | | | D | E | F |
|---|---|---|---|---|---|---|
| | A | B | C | Flexion/ | Varus/valgus | Varus/varus |
| 1. | Condition | # | Cause | Extension angle | extension | flexion |

A.

| | | | | | | |
|---|---|---|---|---|---|---|
| 2. | Tight PCL | 1 | Tight PCL | | dy (me) = dy (le) | dy (mf) = dy (lf) |
| | | | | | medial extension | dy (mf) > dy(me) |
| | | | | | gap = lateral | Medial flexion |
| | | | | | extension gap | gap = lateral |
| | | | | | | flexion gap and |
| | | | | | | flexion gaps > |
| | | | | | | extension gaps- |
| | | | | | | lift off around PCL |
| 3. | Tight medially | 2 | Anterior | | dy (me) = dy (le) | dy (lf) > dy (mf) |
| | in flexion | | MCL | | medial extension | lateral flexion |
| | Loose medially | | | | gap = lateral | gap > medial |
| | in extension | | | | extension gap | flexion gap |
| 4. | Balanced in | 3a | Posterior | fa > 10° | dy (me) = dy (le) | dy (mf) = dy (lf) |
| | flexion | | MCL | flexion | medial extension | dy (lf) > dy (le) |
| | Tight in | | | contraction | gap = lateral | medial flexion |
| | extension | | | | extension gap | gap = lateral |
| | | | | | | flexion gap, and |
| | | | | | | flexion gap is |
| | | | | | | bigger than |
| | | | | | | extension gap |
| 5. | | 3b | Medial | fa > 10° | dy (me) = dy (le) | dy (mf) = dy (lf) |
| | | | posterior | flexion | medial extension | dy (lf) > dy (le) |
| | | | capsule | contraction | gap = lateral | medial flexion |
| | | | | | extension gap | gap = lateral |
| | | | | | | flexion gap, and |
| | | | | | | flexion gap is |
| | | | | | | bigger than |
| | | | | | | extension gap |
| 6. | Tight medially | 4a | Anterior | fa > 10° | dy (me) < dy (le) | dy (mf) < dy (lf) |
| | in flexion | | MCL | flexion | medial extension | medial flexion |
| | Tight medially | | | contraction | gap < lateral | gap < lateral |
| | in extension | | | | extension gap | flexion gap |
| 7. | | 4b | Posterior | | dy (me) < dy (le) | dy (mf) < dy (lf) |
| | | | MCL | | medial extension | medial flexion |
| | | | | | gap < lateral | gap < lateral |
| | | | | | extension gap | flexion gap |
| 8. | | 4c | Medial | | dy (me) < dy (le) | dy (mf) < dy (lf) |
| | | | posterior | | medial extension | medial flexion |
| | | | capsule | | gap < lateral | gap < lateral |
| | | | | | extension gap | flexion gap |
| 9. | | 4d | Semimembranosus | | dy (me) < dy (le) | dy (mf) < dy (lf) |
| | | | and pes anserinus | | medial extension | medial flexion |
| | | | | | gap < lateral | gap < lateral |
| | | | | | extension gap | flexion gap |
| 10. | Tight popliteus | 5 | Popliteus | | | |
| | tendon | | tendon | | | |
| 11. | Compensatory | 6 | Iliotibial | | dy (me) > dy (le) | |
| | lateral release - | | band | | medial extension | |
| | extension only | | | | gap > lateral | |
| | | | | | extension gap | |
| 12. | Compensatory | 7 | LCL and | | dy (me) > dy (le) | dy mf > dy (lf) |
| | lateral release - | | popliteus endon | | medial extension | medial flexion |
| | flexion and | | | | gap > lateral | gap > lateral |
| | extension | | | | extension gap | flexion gap |
| 13. | Tight laterally | 8a | Popliteus | | dy (me) > dy (le) | dy mf > dy (lf) |
| | in flexion | | tendon | | medial extension | medial flexion |
| | Tight laterally | | | | gap > lateral | gap > lateral |
| | in extension | | | | extension gap | flexion gap |
| 14. | | 8b | LCL | | dy (me) > dy (le) | dy mf > dy (lf) |
| | | | | | medial extension | medial flexion |
| | | | | | gap > lateral | gap > lateral |
| | | | | | extension gap | flexion gap |
| 15. | | 8c | Posteralateral | | dy (me) > dy (le) | dy mf > dy (lf) |
| | | | corner of capsule | | medial extension | medial flexion |
| | | | | | gap > lateral | gap > lateral |
| | | | | | extension gap | flexion gap |
| 16. | Tight laterally | 8d | Popliteus | | dy (me) > dy (le) | dy (mf) > dy (lf) |
| | in flexion | | tendon | | medial extension | dy (le) < dy (lf) |
| | Tight laterally | | | | gap > lateral | medial flexion |
| | in extension | | | | extension gap | gap > lateral |
| | (tighter in | | | | | flexion gap and |
| | extension than | | | | | lateral extension |
| | in flexion) | | | | | gap < lateral |
| | | | | | | flexion gap |
| 17. | Balanced in | 9a | Iliotibial | | dy (le) < dy (me) | dy (lf) = dy (mf) |
| | flexion | | band | | lateral extension | lateral flexion |
| | Tight laterally | | | | gap < medial | gap = medial |
| | in extension | | | | extension gap | flexion gap |
| 18. | | 9b | Lateral | | dy (le) < dy (me) | dy (lf) = dy (mf) |
| | | | posterior | | lateral extension | lateral flexion |
| | | | capsule | | gap < medial | gap = medial |
| | | | | | extension gap | flexion gap |
| 19. | Tight laterally | 10a | Popliteus | | dy (me) = dy (le) | dy (lf) < dy (mf) |
| | in flexion | | tendon | | medial extension | lateral flexion |
| | Balanced in | | | | gap = lateral | gap < medial |
| | extension | | | | extension gap | flexion gap |
| 20. | | 10b | LCL | | dy (me) = dy (le) | dy (lf) < dy (mf) |
| | | | | | medial extension | lateral flexion |
| | | | | | gap = lateral | gap < medial |
| | | | | | extension gap | flexion gap |
| 21. | | 10c | Posterolateral | | dy (me) = dy (le) | dy (lf) < dy (mf) |
| | | | corner | | medial extension | lateral flexion |
| | | | of capsule | | gap = lateral | gap < medial |
| | | | | | extension gap | flexion gap |
| 22. | Deficient PCL | 11 | PCL | | | |

B.

| | | | | | | |
|---|---|---|---|---|---|---|
| 2. | Tight PCL | 1 | Tight PCL | dz (me) = dz (le) | dz (mf) > 0 > TBD | |
| | | | | posterior medial | dz (mf) > dz (lf) | |
| | | | | rollback in | medial femoral | |
| | | | | extension = | rollback is | |
| | | | | posterior lateral | posterior, TBD value | |
| | | | | rollback in | determines how far | |
| | | | | extension | beyond midline, and | |
| | | | | | medial rollback > | |
| | | | | | posterior | |
| | | | | | lateral rollback | |
| 3. | Tight medially | 2 | Anterior | dz(me) = dz(le) | dz (mf) > 0 > TBD | ri (me) < re (le) |
| | in flexion | | MCL | posterior medial | dz (mf) > dz (lf) | Internal rotation |
| | Loose medially | | | rollback in | medial femoral | about the medial |
| | in extension | | | extension = | rollback is | complex is < |
| | | | | posterior lateral | posterior, TBD value | external rotation |
| | | | | rollback in | determines how far | about the lateral |
| | | | | extension | beyond midline, and | complex in |
| | | | | | medial rollback > | extension |
| | | | | | posterior | |
| | | | | | lateral rollback | |
| 4. | Balanced in | 3a | Posterior | dz(le) = dz(me) | dz(le) < dz(lf) | |
| | flexion | | MCL | posterior medial | dz(me) < dz(mf) | |
| | Tight in | | | rollback = | lateral and | |
| | extension | | | posterior lateral | medial rollback | |
| | | | | rollback in | in extension are | |
| | | | | extension | less than lateral | |
| | | | | | and medial | |
| | | | | | rollback in flexion | |
| 5. | | 3b | Medial | dz (le) = dz (me) | dz(le) < dz(lf) | |
| | | | posterior | posterior medial | dz(me) < dz(mf) | |
| | | | capsule | rollback = | lateral and | |
| | | | | posterior lateral | medial rollback | |
| | | | | rollback in | in extension are | |
| | | | | extension | less than lateral | |
| | | | | | and medial | |
| | | | | | rollback in flexion | |
| 6. | Tight medially | 4a | Anterior | dz(me) < dz(le) | dz(mf) < dz(lf) | ri (mf) < re (lf) |
| | in flexion | | MCL | posterior medial | posterior medial | internal rotation |
| | Tight medially | | | rollback < | rollback < | about medial |
| | in extension | | | posterior lateral | posterior lateral | side in flexion < |
| | | | | rollback in | rollback in flexion | external rotation |
| | | | | extension | | about the lateral side |
| 7. | | 4b | Posterior | dz (me) < dz (le) | dz (mf) < dz (lf) | ri (mf) < re (lf) |
| | | | MCL | posterior medial | posterior medial | internal rotation |
| | | | | rollback < | rollback < | about medial |
| | | | | posterior lateral | posterior lateral | side in flexion < |
| | | | | rollback in | rollback in flexion | external rotation |
| | | | | extension | | about the lateral side |
| 8. | | 4c | Medial | dz (me) < dz (le) | dz (mf) < dz (lf) | ri (mf) < re (lf) |
| | | | posterior | posterior medial | posterior medial | internal rotation |
| | | | capsule | rollback < | rollback < | about medial |
| | | | | posterior lateral | posterior lateral | side in flexion < |
| | | | | rollback in | rollback in flexion | external rotation |
| | | | | extension | | about the lateral side |
| 9. | | 4d | Semimembranosus | dz (me) < dz (le) | dz (mf) < dz (lf) | ri (mf) < re (lf) |
| | | | and pes | posterior medial | posterior medial | internal rotation |
| | | | anserinus | rollback < | rollback < | about medial |
| | | | | posterior lateral | posterior lateral | side in flexion < |
| | | | | rollback in | rollback in flexion | external rotation |
| | | | | extension | | about the lateral side |
| 10. | Tight popliteus | 5 | Popliteus | | | ri (mf) > re (lf) |
| | tendon | | tendon | | | internal rotation |
| | | | | | | about medial |
| | | | | | | side > external |
| | | | | | | rotation about |
| | | | | | | lateral side |
| 11. | Compensatory | 6 | Iliotibial | | | |
| | lateral release - | | band | | | |
| | extension only | | | | | |
| 12. | Compensatory | 7 | LCL and | | | |
| | lateral release - | | popliteus | | | |
| | flexion and | | tendon | | | |
| | extension | | | | | |
| 13. | Tight laterally | 8a | Popliteus | dz(me) > dz(le) | dz(mf) > dz(lf) | ri(me) > re(le) |
| | in flexion | | tendon | posterior medial | posterior medial | internal rotation |
| | Tight laterally | | | rollback > | rollback > | about the medial |
| | in extension | | | posterior lateral | posterior lateral | side > external |
| | | | | rollback in | rollback in flexion | rotation about |
| | | | | extension | | the lateral side |
| 14. | | 8b | LCL | dz(me) dz (le) | dz (mf) > dz (lf) | ri (me) > re (le) |
| | | | | posterior medial | posterior medial | internal rotation |
| | | | | rollback > | rollback > | about the medial |
| | | | | posterior lateral | posterior lateral | side > external |
| | | | | rollback in | rollback in flexion | rotation about |
| | | | | extension | | the lateral side |
| 15. | | 8c | Posteralateral | dz (me) > dz (le) | dz (mf) > dz (lf) | ri (me) > re (le) |
| | | | corner | posterior medial | posterior medial | internal rotation |
| | | | of capsule | rollback > | rollback > | about the medial |
| | | | | posterior lateral | posterior lateral | side > external |
| | | | | rollback in | rollback in flexion | rotation about |
| | | | | extension | | the lateral side |
| 16. | Tight laterally | 8d | Popliteus | dz (me) > dz (le) | dz (mf) > dz (lf) | ri (me) > re (le) |
| | in flexion | | tendon | posterior medial | dz (le) < dz (lf) | internal rotation |
| | Tight laterally | | | rollback > | posterior medial | about the medial |
| | in extension | | | posterior lateral | rollback > | side > external |
| | (tighter in | | | rollback in | posterior lateral | rotation about |
| | extension than | | | extension | rollback in | the lateral side |
| | flexion) | | | | flexion and | |
| | | | | | posterior lateral | |
| | | | | | rollback in | |
| | | | | | extension > | |
| | | | | | posterior lateral | |
| | | | | | rollback in flexion | |
| 17. | Balanced in | 9a | Iliotibial | dz (le) < dz (me) | dz (lf) = dz (mf) | ri (le) < re (me) |
| | flexion | | band | posterior lateral | posterior lateral | internal rotation |
| | Tight laterally | | | rollback < | rollback = | about lateral |
| | in extension | | | posterior medial | posterior medial | side < external |
| | | | | rollback in | rollback in flexion | rotation about |
| | | | | extension | | medial side |
| 18. | | 9b | Lateral | dz (le) < dz (me) | dz (lf) = dz (mf) | ri (le) < re (me) |
| | | | posterior | posterior lateral | posterior lateral | internal rotation |
| | | | capsule | rollback < | rollback = | about lateral |
| | | | | posterior medial | posterior medial | side < external |
| | | | | rollback in | rollback in flexion | rotation about |
| | | | | extension | | medial side |
| 19. | Tight laterally | 10a | Popliteus | dz (le) = dz (me) | dz (lf) < dz (mf) | |
| | in flexion | | tendon | posterior lateral | posterior lateral | |
| | Balanced in | | | rollback = | rollback < | |
| | extension | | | posterior medial | posterior medial | |
| | | | | rollback in | rollback in flexion | |
| | | | | extension | | |
| 20. | | 10b | LCL | dz (le) = dz (me) | dz (lf) < dz (mf) | |
| | | | | posterior lateral | posterior lateral | |
| | | | | rollback = | rollback < | |
| | | | | posterior medial | posterior medial | |
| | | | | rollback in | rollback in flexion | |
| | | | | extension | | |
| 21. | | 10c | Posterolateral | dz (le) = dz (me) | dz (lf) < dz (mf) | |
| | | | corner | posterior lateral | posterior lateral | |
| | | | of capsule | rollback = | rollback < | |
| | | | | posterior medial | posterior medial | |
| | | | | rollback in | rollback in flexion | |
| | | | | extension | | |
| 22. | Deficient PCL | 11 | PCL | | dz (lf) < 0 | ri (me) < re (le) |
| | | | | | dz (mf) < 0 | internal rotation |
| | | | | | medial and | about medial |
| | | | | | lateral condyles | side < external |
| | | | | | are displaced | rotation about |
| | | | | | negatively (i.e., | lateral side |
| | | | | | anteriorly) | |

For navigating surgical instrument, prosthetic components, and other items, the systems and processes according to an embodiment of the present invention can invoke and employ various navigational algorithms, either commercially available or proprietary. In one embodiment illustrated in FIG. 3, the proprietary “AchieveCAS” TKA software is used in this capacity.

As illustrated in FIG. 1, systems according to some embodiments of the present invention may also comprise an imager for obtaining at least one image of the tibia, the femur, the tibial prosthetic component, or the femoral prosthetic component, wherein the computer is adapted to receive from the imager and store the at least one image of the tibia, the femur, the tibial prosthetic component, or the femoral prosthetic component; and a monitor adapted to receive information from the computer in order to display the at least one image of the tibia, the femur, the tibial prosthetic component, or the femoral prosthetic component.

Systems according to some embodiments may further comprise surgical instruments associated with one or more fiducials and adapted for navigation and positioning at the knee using the images displayed on the monitor. The systems may further comprise prosthetic components associated with one or more fiducials and adapted for navigation and positioning at the knee using the images displayed on the monitor. The systems may further comprise at least one cutting jig or cutting block for positioning at the femur, wherein the cutting jig is associated with one or more fiducials and the position and orientation of the fiducial associated with the cutting jig is trackable by the computer for navigation and positioning of the cutting jig at the femur. The cutting jig or block may be adjustable and/or multi-purpose.

The systems and processes according to aspects and embodiments of the present invention can be adapted the variety of the surgical techniques and surgeon's preferences. The systems and processing according to the embodiments of the present invention employ surgeon profiles so that the surgeon can retrieve his or her surgical setup or profile from the computer memory. However, the user, such as the surgeon, can change the setup before, after or during the surgery to incorporated desired changes needed based on surgical anatomy, and/or anomalies specific to a patient, or a prosthetic device. This system provides objective measures assess the soft-tissue balancing within TKA by applying a logic matrix to the data acquired during the static assessment and the kinematic testing of the knee joint. The systems and processes are flexible and can be adapted to the technique-employed by the surgeon. The systems can also be used to verify implant trial placement when using conventional surgical TKA techniques. The logic matrix is programmable and can be adapted to the individual needs of the surgeon. For example, the system can be adapted to allow the surgeon to modify the default threshold values, and add to or delete information from the logic matrix. Some embodiments of the invention can also provide a method of computer-assisted total arthroplasty on a patient's knee using the above-described systems and processes.

### A Soft-Tissue Balancing Algorithm Based on the Convergence of the Anatomical Landmarks and the Dynamic Interaction of the Knee bones and Ligaments

In one embodiment of the present invention, the systems, methods and processes employ a soft-tissue balancing algorithm that advantageously considers and correlates both the anatomical landmarks and the dynamic interaction of the knee bones and ligaments, an important advantage over the existing methods that are generally excessively weighted towards either anatomical or dynamic factors. The algorithm also advantageously considers and correlates both femoral and tibial landmark, an advantage over the existing methods that commonly consider only femoral or only tibial landmarks. The method establishes a rectangular gap between tibia and femur in both flexion and extension without distorting the anatomy of the knee. According to some aspects and embodiments of the method, prosthetic component size, positioning, and surgical cuts can be planned before any irreversible bone cuts are made, although the system and method are adaptable for ligament balancing in patients after the surgical cuts are performed, or after the prosthetic components are installed. It is to be understood that the method is performed with the computer assistance and in the context of computer-assisted surgical systems and methods as described elsewhere herein. Consideration of the anatomy, kinematics, coordinate systems, and of real and/or virtual surgical constructs, such as axes and planes generally involves storage of data in computer memory and calculations optimally performed with the aid of a computer. A computer-assisted surgical system according to some embodiments of the present invention employs computers programmed with the algorithms for performing the steps necessary for carrying out the method.

With reference to FIGS. 2, and 8-9, the method can be used as follows. The surgeon exposes the knee in a conventional manner, and performs preliminary osteophyte resection and ligament release. The anterior cruciate ligament may be divided, if present, and/or the posterior cruciate ligament may be resected at the surgeon's discretion. The distal femoral anatomy is registered by the imager and digitized and the proposed position of the femoral component based on the traditional anatomical landmarks, such as a posterior condylar or epicondylar rotation and posterior condylar measured resection are registered. FIG. 2 shows an exposed human knee (200) in a surgical field after the osteophyte resection and the preliminary ligament release. The surgeon establishes femoral and tibial coordinate systems by, for example, registering the navigational landmarks for the end of the respective bones. The navigation instrument (202) on the distal femur (206) tracks the position of the femur relative to the tibial coordinate system. The femur is distracted in flexion and extension.

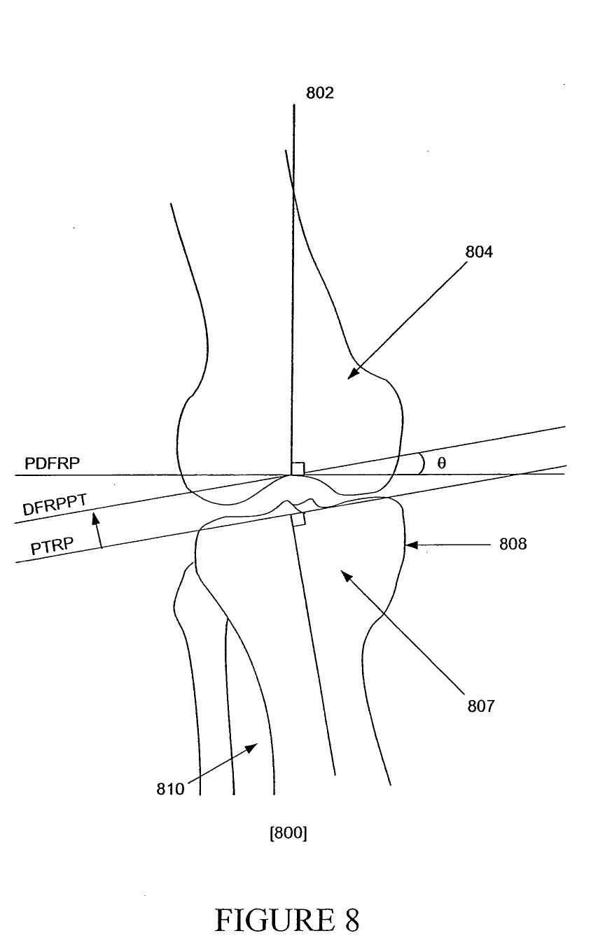

As shown in FIG. 8, with the knee (800), comprising femur (804), tibia (808) and fibula (810),

in extension, a proposed distal femoral resection plane perpendicular to the mechanical axis (802) of the femur (804) in varus/valgus (PDFRP; an anatomical femoral resection plane) is established, and a proposed tibial resection plane (PTRP) perpendicular to the mechanical axis (807) of the tibia (808) in varus/valgus is established. Using a navigation instrument on the distal femur shown in FIG. 2 (204), tracking its position relative to the tibial coordinate system, distal femoral resection plane is established that is perpendicular to the long axis of the tibia (DFRPPT, a dynamic resection plane). Using the anatomical landmarks the femoral resection plane perpendicular to the tibia (DFRPPT) is compared to the proposed femoral resection plane perpendicular to the mechanical axis of the femur (PDFRP). The state of the soft tissue balance of the knee is represented in extension by the angle 0 formed between the femoral anatomical resection plane and the femoral dynamic resection planes in extension.

In one embodiment, the final femoral resection level is not determined until after the soft tissues are balanced. To perform the resection using computer-assisted navigation, the pins are placed in the distal femur for positioning of a distal femoral cutting jig at a known angle to the mechanical axis of the femur.

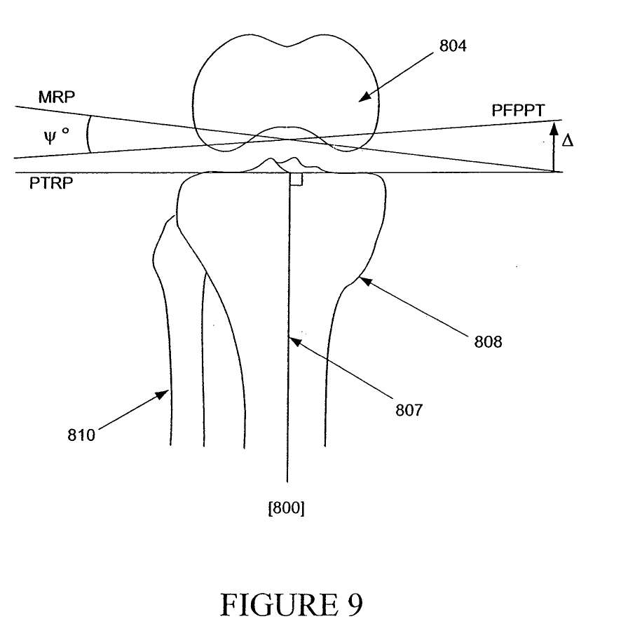

As shown in FIG. 9, when the knee (800) is flexed, a proposed posterior femoral resection plane perpendicular to the mechanical axis (802) of the femur (804) is established (MRP; an anatomical femoral resection plane), a proposed tibial resection plane (PTRP) perpendicular to the mechanical axis of the tibia (806) in varus/valgus distraction, and posterior femoral resection plane perpendicular to the mechanical axis of the tibia (PFPPT, a dynamic resection plane) are established. Using the anatomical landmarks, PFPPT is compared in to MRP. The state of the soft tissue balance of the knee (800) is represented in flexion by the angle φ formed between the femoral anatomical resection plane and the femoral dynamic resection plane.

In flexion and extension, if the anatomical and the femoral resection planes agree, they are approximately parallel and the angles φ and θ are close to 0. The resection gap in the knee is then approximately rectangular in both flexion and extension. If not, more soft tissue balancing, such as ligament release and contraction, is necessary. Based on the angle, the system establishes if the ligaments need further adjustment, and provide necessary recommendations to the surgeon on ligament balancing. For example, as shown in FIG. 8, the medial side of the knee is tight and the planes are at a non-zero angle θ. Based on the calculated angle θ, the system employing the provided method suggests that the medial side is tight in extension and may need further released. Upon soft tissue adjustment, the state of the knee is reassessed. The distance Δ between the tibial and the femoral resection planes preferably allows for placement of the tibial tray, plastic femur, and bone cement.

The iterative cycle of knee assessment and ligament balancing is performed until the anatomical and the dynamic planes converge. It is to be appreciated that convergence does not necessarily mean coincidence, and that the known principles of the iterative convergence methods and their limitations are utilized in the embodiments of the present invention.

The bones can be resected at the recommended converged planes, or an existing surgical plane may be assigned to the algorithm. Due to the fact that ligament balancing and surgical planes prediction according to certain aspects and embodiments of the method occur prior to resection of the leg bones, the method facilitates minimally invasive, small-incision TKR. The adjustable and/or multifunctional cutting jigs or blocks can be used in conjunction of the method of the present application.

The method can be adapted to various special circumstances. For example, in case of significant flexion constructure, preliminary distal femoral and posterior femoral cuts may be necessary to remove posterior osteophytes and ensure adequate posterior capsule release. In general, the preliminary resection may be shallow enough so as not to determine the final surgical cutting planes in accordance with the provided method and algorithm. The method can be adapted to particular prosthetic systems and methods of installation thereof. For example, certain available knee prosthetic components are adapted for placement at pre-determined angles to the tibial and femora axes. Such features of the prosthetic systems are easily incorporated into the provided method by assigning appropriate parameters.

The foregoing discloses preferred embodiments of the present invention, and numerous modifications or alterations may be made without departing from the spirit and the scope of the invention.

The particular embodiments of the invention have been described for clarity, but are not limiting of the present invention. Those of skill in the art can readily determine that additional embodiments and features of the invention are within the scope of the appended claims and equivalents thereto. All publications cited herein are incorporated by reference in their entirety.

1. A system for use by a surgeon in the course of computer-assisted total arthroplasty on a patient's knee. The system comprises:
at least one first fiducial associated with a femur;
at least one second fiducial associated with a tibia;
a tracking functionality capable of tracking a position and orientation of the at least one first fiducial and the at least one second fiducial;
a computer, wherein the computer is
adapted to receive and store information from the tracking functionality on the position and orientation of the at least one first fiducial and the at least one second fiducial,
adapted to acquire information during kinematic testing relating to the position and orientation of the at least one first fiducial and the at least one second fiducial;
adapted to store in memory a knee variable determined based on an anatomical method and a knee variable based on a dynamic method;
adapted to assess the difference between the variable based on the anatomical method and the variable based on the dynamic method; and
adapted to provide the recommendations to the surgeon on adjustment of the soft tissues of the knee with the purpose of reducing the difference between the variable based on the anatomical method and the variable based on the dynamical method.

2. The system of claim 1, further comprising:
an imager for obtaining at least one image of the tibia or the femur, wherein the computer is adapted to receive from the imager and store the at least one image of the tibia or the femur; and
a monitor adapted to receive information from the computer in order to display the at least one image of the tibia or the femur.

3. The system of claim 1, further comprising a surgical instrument associated with one or more fiducials and adapted for navigation and positioning at the knee, wherein the one or more fiducials associated with the instruments are adapted to be tracked by the tracking functionality.

4. The system of claim 1 further comprising a prosthetic component associated with one or more fiducials and adapted for navigation and positioning at the knee, wherein the one or more fiducials associated with the prosthetic component are adapted to be tracked by the tracking functionality

5. The system of claim 1, further comprising a cutting guide for positioning at the femur or the tibia, wherein the cutting guide is associated with one or more fiducials, and the one or more fiducials associated with the cutting jig are adapted to be tracked by the tracking functionality.

6. The system of claim 5, wherein the position of the cutting guide at the femur or the tibial is be adjustable in at least one degree of rotational or at least one degree of translational freedom.

7. A method of computer-assisted total arthroplasty on a patient's knee, comprising the steps of:
registering with a computer at least one first fiducial associated with the femur; and at least one second fiducial associated with the tibia;
tracking position and orientation of the at least one first fiducial and the at least one second fiducial with a tracking functionality;
using the computer adapted to receive signals and store information from the tracking functionality on the position and orientation of the at least one first fiducial and thus the femur; and the at least one second fiducial and thus the tibia;
using the computer to store in memory a knee variable determined based on an anatomical method and a knee variable based on a dynamic method;
using the computer to assess the difference between the variable based on the anatomical method and the variable based on the dynamic method; and
using the computer to provide the recommendations to the surgeon on adjustment of the soft tissues of the knee with the purpose of reducing the difference between the variable based on the anatomical method and the variable based on the dynamical method.

8. The method of claim 7, further comprising positioning a cutting guide at the femur; and resecting the femur based on the recommendations.

9. The method of claim 7, further comprising the steps of:
using an imager for obtaining at least one image of a tibia or a femur, wherein the computer is adapted to receive from the imager and store the at least one image of the tibia or the femur; and
using a monitor adapted to receive information from the computer to display the at least one image of the tibia or the femur.

10. The method of claim 7, further comprising the step of registering with the computer and navigating and positioning at the knee of a surgical instrument associated with one or more fiducials.

11. The method of claim 7, further comprising the step of registering with the computer and navigating and positioning at the knee of prosthetic components associated with one or more fiducials.

12. The method of claim 7, further comprising the steps of registering with the computer and navigating and positioning at the femur or the tibia of a cutting guide associated with one or more fiducials.

13. The method of claim 7, wherein a position of the cutting guide at the femur or the tibia is adjustable at the femur or the tibia in at least one degree of rotational or at least one degree of translational freedom.

14. The method of claim 7, further comprising the step of using the computer to provide recommendations on selecting a prosthetic component at the knee.

15. The method of claim 7, further comprising the step of using the computer to provide recommendations on positioning a prosthetic component at the knee.

16. The method of claim 7, further comprising adjusting the soft tissues of the knee.

17. The method of claim 7, wherein adjusting the soft tissues of the knee comprises at least one of releasing or contracting ligaments.

18. A method of computer-assisted soft tissue balancing in a knee during total knee arthroplasty, comprising the steps of:
a. providing a computer comprising a memory, and a tracking functionality, wherein the tracking functionality is adapted to track position and orientation of fiducials and transmitting information on the position and orientation of the fiducials to the computer functionality;
b. associating at least one first fiducial with a femur;
c. associating at least one second fiducial with a tibia;
d. registering the femur and the tibia with the computer;
e. establishing in the computer memory femoral and tibial coordinate systems;
f. establishing in the computer memory a mechanical axis of the femur and a mechanical axis of the tibia;
g. establishing in the computer memory a femoral resection plane perpendicular to the mechanical axis of the femur, and a proposed tibial resection plane perpendicular to a mechanical axis of the tibia;
h. distracting the knee in flexion and extension in at least one of varus/valgus, AP drawer, or rotation tests, and establishing in a computer memory femoral resection planes perpendicular to the long axis of the tibia in flexion and extension;
i. calculating an angle between the femoral resection planes perpendicular to the long axis of the tibia to the femoral resection planes perpendicular to the mechanical axis of the femur, whereby state of the soft tissue balance of the knee is represented in flexion and extension by the angle;
j. using the computer to provide recommendations to the surgeon on adjustment of soft tissues with the purpose of reducing the angle;
k. adjusting the soft tissues; and
l. repeating the steps h through k until the femoral resection planes perpendicular to the long axis of the tibia to the femoral resection planes perpendicular to the mechanical axis of the femur converge.

19. The method of claim 18, further comprising using the computer to provide recommendations on femoral cutting planes based on convergence of planes in step l.

20. The method of claim 18, further comprising positioning a cutting guide at the femur; and resecting the femur based on the recommendations.
